(19) United States
(12) Patent Application Publication
(10) Pub. No.: US 2008/0168596 A1
(43) Pub. Date: Jul. 17, 2008

(54) DRAIN STOPPER WITH DEODORIZER

(76) Inventors: John A. Findlay, Mission Viejo, CA (US); Michelle D. James, Rancho Santa Margarita, CA (US); Guy D. Gardner, Placentia, CA (US)

Correspondence Address: STETINA BRUNDA GARRED & BRUCKER, 75 ENTERPRISE, SUITE 250, ALISO VIEJO, CA, 92656, US

(21) Appl. No.: 12/016,027

(22) Filed: Jan. 17, 2008

Related U.S. Application Data
(60) Provisional application No. 60/885,358, filed on Jan. 17, 2007.

(51) Int. Cl. A47K 1/14; A61L 9/015; A61L 9/04

(52) U.S. Cl. 4/287; 424/76.2; 239/60

(57) ABSTRACT

A drain stopper adapted for retrofit application to the drain assembly of an existing sink, tub or other water retaining basin. The drain stopper of the present invention comprises an elongate connecting rod having an end cap threadably engaged to one end thereof. Also cooperatively engaged to the connecting rod is a filter basket, one open end of which is enclosed by the threadable engagement of the end cap to the connecting rod. The selective detachment of the end cap from the connecting rod allows the interior of the filter basket to be filled with a suitable deodorizer. The filter basket may optionally be provided with a plurality of projections which are sized and configured to capture hair or other debris passing into a drain opening in which the drain stopper is positioned.

### CROSS-REFERENCE TO RELATED APPLICATIONS

The present application claims priority to U.S. Provisional Patent Application Ser. No. 60/885,358 entitled DRAIN STOPPER WITH DEODORIZER filed Jan. 17, 2007.

### STATEMENT RE: FEDERALLY SPONSORED RESEARCH/DEVELOPMENT

Not Applicable

### BACKGROUND OF THE INVENTION

1. Field of the Invention

The present invention relates generally to plumbing fixtures, and more particularly to a uniquely configured pop-up drain stopper which is adapted for retrofit application to the drain assembly of an existing sink, tub or other water retaining basin, and is outfitted with a deodorizer for minimizing the escape of undesirable odors from the drain opening of the basin.

2. Description of the Related Art

As is well known in the plumbing industry, conventional pop-up drain stopper assemblies typically comprise a stopper which itself includes a button-like cap having a connecting rod extending generally axially therefrom. Extending about the periphery of the cap is a sealing member, such as an O-ring. The distal end of the connecting rod typically defines an opening or through hole which is adapted to receive one end of an elongate pivoting lever arm or lift rod. In typical drain stopper assemblies, the lift rod is advanced through a pivot nut which protrudes from the drain pipe extending downwardly from the drain opening of a water retaining basin such as a sink with which the drain stopper assembly is used. Upon being advanced through the pivot nut, one end of the lift rod is cooperatively engaged to the connecting rod of the stopper in the above-described manner, with the opposite end of the lift rod being cooperatively engaged to a linkage. Such linkage is in turn cooperatively engaged to the lower end of an elongate stem or handle. A portion of the stem or handle protrudes upwardly from the sink in close proximity to the associated faucet handles to allow the same to be easily grasped by a user.

In operation, due to the pivotal interface of the lift rod to the drain pipe via the pivot nut, pulling the stem upwardly results in the pivoting of the lift rod in a manner which draws the stopper downwardly into the drain pipe. The downward movement of the stopper is limited by the engagement of the sealing member of the cap thereof into the complimentary drain opening of the sink. Conversely, the application of downward pressure to the stem facilitates the pivotal movement of the lift rod in a manner vertically translating the stopper upwardly and lifting the cap of the stopper out of its sealed engagement within the drain opening. Once the cap of the stopper is lifted out of sealed engagement within the drain opening, water is free to drain from the sink into the drain pipe via the drain opening, the draining water still flowing about the connecting rod of the stopper and that portion of the lift rod advanced into the drain pipe.

Despite the existence of traps within the drain pipes of sinks and other water retaining basins, it is not uncommon for undesirable odors to emanate from the drain opening. Currently known pop-up drain stopper assemblies, though being suitable to selectively close the drain opening of the sink or other basin, are not outfitted with any modality which assists in minimizing or eliminating odors emanating from the drain opening when the cap of the stopper is lifted out of sealed engagement to the drain opening. The present invention addresses this shortcoming by providing a drain stopper which may be retrofit to an existing drain stopper assembly and is provided with a deodorizer which eliminates or minimizes the undesirable emanation of odor from the drain opening. These, as well as other features and advantages attendant to the present invention will be discussed in more detail below.

### BRIEF SUMMARY OF THE INVENTION

In accordance with the present invention, there is provided a first embodiment of a drain stopper adapted for retrofit application to the drain assembly of an existing sink, tub or other water retaining basin. The drain stopper of the present invention comprises an elongate connecting rod having an end cap threadably engaged to one end thereof. Also cooperatively engaged to the connecting rod is a filter basket, one open end of which is enclosed by the threadable engagement of the end cap to the connecting rod. The selective detachment of the end cap from the connecting rod allows the interior of the filter basket to be filled with a suitable deodorizer. In accordance with a second embodiment of the present invention, the filter basket is provided with a plurality of projections which are sized and configured to capture hair or other debris passing into a drain opening in which the drain stopper is positioned.

### BRIEF DESCRIPTION OF THE DRAWINGS

These, as well as other features of the present invention, will become more apparent upon reference to the drawings wherein:

FIG. 1 is a cross-sectional view of a conventional sink as retrofitted to include the deodorizing drain stopper constructed in accordance with the present invention;

FIG. 2 is a cross-sectional view of the drain stopper of the present invention;

FIG. 3 is a cross-sectional view of the drain stopper of the present invention taken along line 3-3 of FIG. 2;

FIG. 4 is a side-elevational view of the drain stopper of the present invention taken along line 4-4 of FIG. 2; and

FIG. 5 is a side-elevational view of a deodorizing drain stopper constructed in accordance with a second embodiment of the present invention.

Common reference numerals are used throughout the drawings and detailed description to indicate like elements.

### DETAILED DESCRIPTION OF THE INVENTION

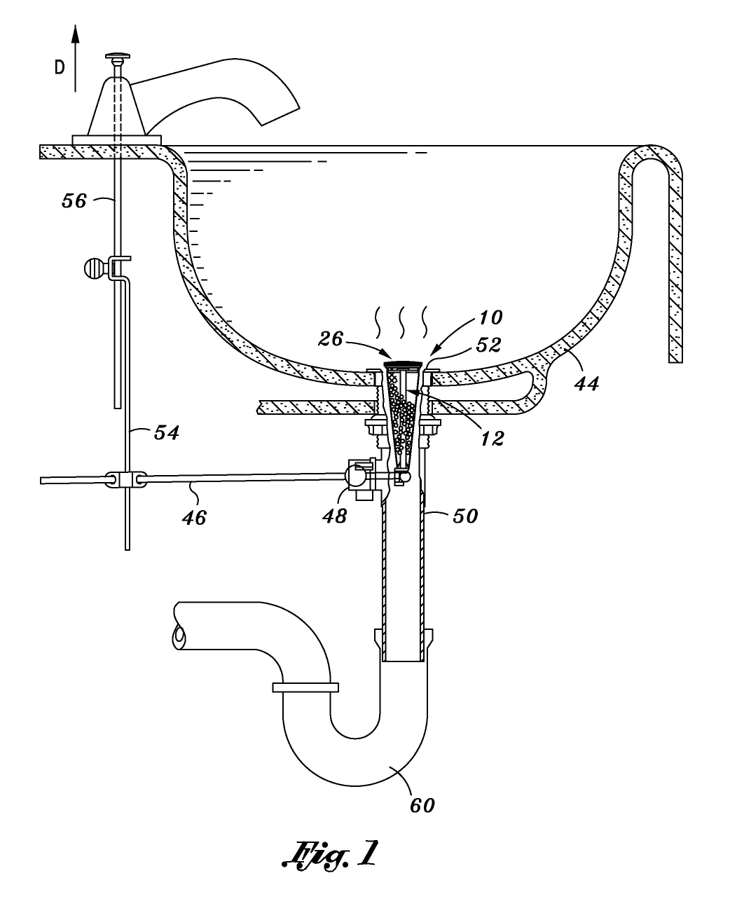

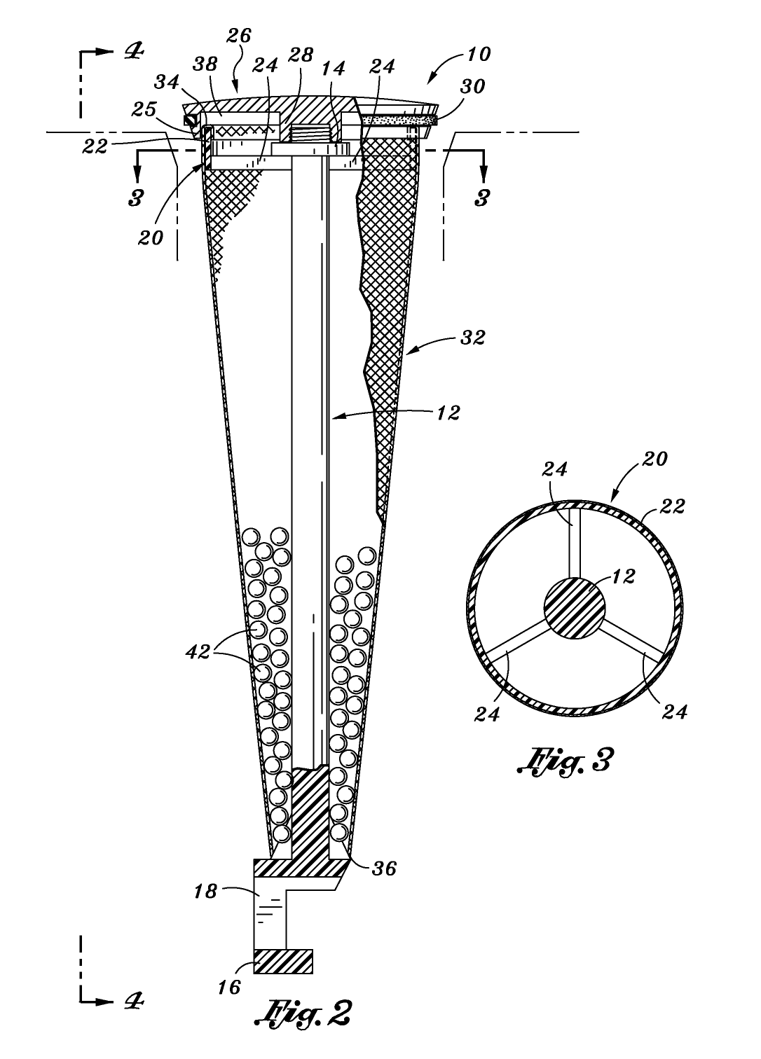

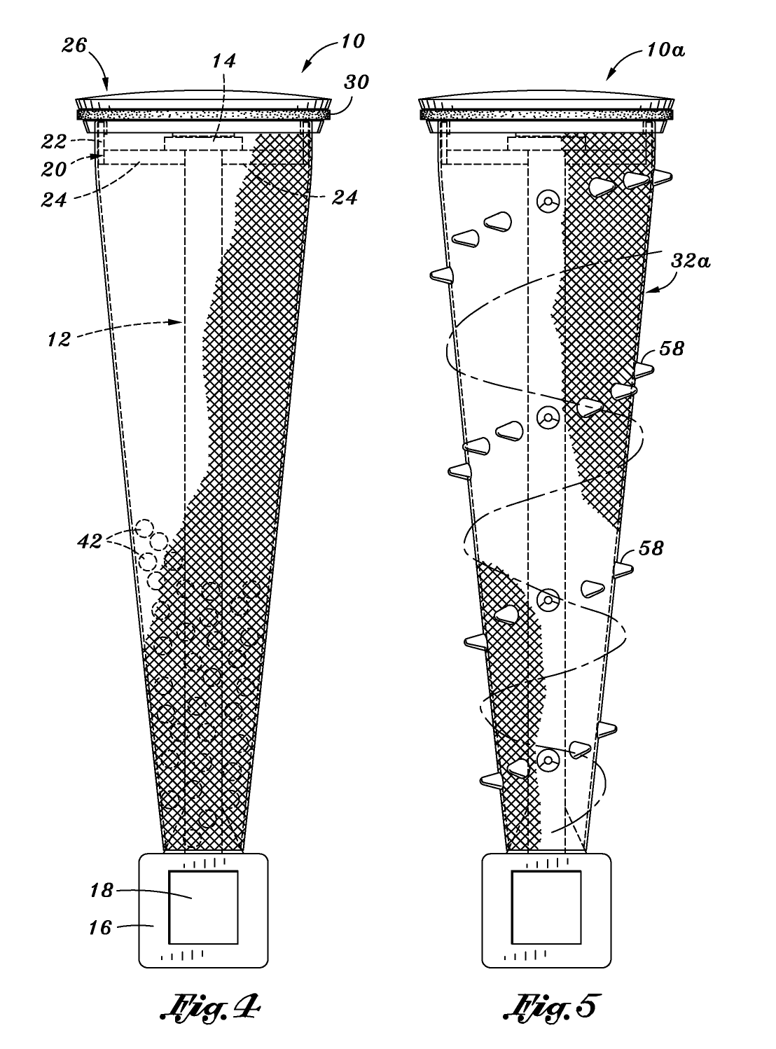

Referring now to the drawings wherein the showings are for purposes of illustrating preferred embodiments of the present invention only, and not for purposes of limiting the same, FIGS. 1-4 depict a deodorizing drain stopper 10 constructed in accordance with the present invention. The drain stopper 10 comprises an elongate connecting rod 12 which may be fabricated from a suitable plastic or metal material. As best seen in FIG. 2, the connecting rod 12 defines a circularly configured flange portion 14 which extends radially outward therefrom in close proximity to one of the opposed ends thereof. In this regard, a portion of the outer surface of the connecting rod 12 extending between the flange portion 14 and that end of the connecting rod 12 disposed closest to the flange portion 14 is externally threaded, as also shown in FIG. 2. The end of the connecting rod 12 opposite that which is externally threaded includes an integral, tubular sleeve portion 16 which defines an opening 18. As shown in FIG. 4, the opening 18 has a generally quadrangular configuration, though those of ordinary skill in the art will recognize that alternative shapes for the opening 18 are contemplated to be within the spirit and scope of the present invention.

The drain stopper 10 further comprises a support frame 20 which is best shown in FIG. 3, and which is cooperatively engaged to the connecting rod 12. The support frame 12 comprises an annular, circularly configured outer wall and three (3) elongate spokes 24 which are integrally connected to the outer wall and extend radially inwardly therefrom at intervals of approximately 120°. The inner end of each of the spokes 24 contacts and is attached to the connecting rod 12 at a location immediately below the flange portion 14 thereof. It is contemplated that the support frame 20 may be integrally connected to the connecting rod 12 and thus comprise an integral portion thereof, as does the flange portion 14 and the sleeve portion 16. Though as depicted in FIG. 3 the support frame 20 includes three spokes 24, those of ordinary skill in the art will recognize that the fabrication of the support frame 20 to include greater or fewer then three spokes 24 arranged at intervals differing from that described above is contemplated to be within the spirit and scope of the present invention.

The drain stopper 10 of the present invention further comprises a button-like cap 26 which is cooperatively engaged to the connecting rod 12. As best seen in FIG. 2, the cap 26 defines an internally threaded, axially extending hub portion 28 which is threadably engageable to the externally threaded portion of the connecting rod 12 in the manner also shown in FIG. 2. In this regard, the advancement of the hub portion 28 along the connecting rod 12 is limited by the abutment or engagement of the distal rim of the hub portion 28 to the flange portion 14 of the connecting rod 12. Formed in the outer surface of a peripheral side wall 25 of the circularly configured cap 26 is a continuous groove or channel which is sized and configured to partially accommodate a sealing O-ring 30 of the drain stopper 10. It is contemplated that the cap 26 will be fabricated from a suitable corrosion resistant metal material.

The drain stopper 10 of the present invention further comprises a hollow, generally conical filter basket 32 which may be fabricated from a porous, mesh-like material of metal or plastic. The filter basket 32 may also be fabricated from a sheet of metal or plastic having a plurality of apertures disposed therein. The filter basket 32 defines an annular, circularly configured upper rim 34 which is advanced over and secured to the outer surface of the outer wall 22 of the support frame 20 in the manner shown in FIG. 2. It is contemplated that a plurality of different attachment methods, including the use of an adhesive, may be used to facilitate the rigid engagement of that portion of the filter basket 32 adjacent the upper rim 34 thereof to the outer surface of the outer wall 22 of the support frame 20. Both the upper rim 34 of the filter basket 32 and the top edge of the outer wall 22 of the support frame 20 which is substantially flush or continuous with the upper rim 34 are advanced into a recess 38 defined by the cap 26 when the cap 26 is threadably engaged to the connecting rod 12. Such recess 38 extends between the hub portion 28 of the cap 26 and the peripheral side wall 25 thereof.

In addition to the upper rim 34, the filter basket 32 defines an annular, circularly configured lower rim 36 which is secured to the sleeve portion 16 of the connecting rod 12 in the manner also shown in FIG. 2. Such attachment may also be facilitated through the use of a suitable adhesive. The filter basket 32 has a tapered configuration, such that the diameter of the upper rim 34 substantially exceeds the diameter of the lower rim 36. Due to the tapered configuration of the filter basket 32, the open space defined between the connecting rod 12 and the filter basket 32 becomes progressively narrower as it extends toward the sleeve potion 16 of the connecting rod 12.

In the drain stopper 10 of the present invention, the interior of the filter basket 32 is accessible by the selective detachment of the cap 26 from the connecting rod 12. As will be recognized, such detachment is facilitated by unscrewing the cap 26 from the connecting rod 12 through the rotation of the cap 26 in a counter-clockwise direction. The detachment of the cap 26 from the connecting rod 12 allows the interior of the filter basket 32 to be filled with a suitable deodorizer, such as the deodorizing beads or pellets 42 depicted in FIGS. 1, 2 and 4. Those of ordinary skill in the art will recognize that the deodorizer placed into the interior of the filter basket 32 may be in a form other than for the above-described pellets 42. In this regard, all that is necessary is that the deodorizer be in a form which allows the same to fill or occupy the majority of the available interior volume of the filter basket 32. Subsequent to the placement of the pellets 42 are other deodorizer into the interior of the filter basket 32, it is contemplated that the cap 26 will be threadably reattached to the connecting rod 12 to assume the position shown in FIGS. 2 and 4.

As indicated above, one of the most common environments in which the drain stopper 10 may be used is with the pop-up drain stopper assembly used in conjunction with a water retaining basin, such as the conventional sink 44 shown in FIG. 1. The drain stopper 10 of the present invention is adapted for retrofit integration into the existing drain stopper assembly of the sink 44, replacing the existing drain stopper thereof. The drain stopper assembly of the sink 44 comprises an elongate pivoting lever arm or lift rod which is advanced through a pivot nut 48 protruding from the drain pipe 50 which extends downwardly from the drain opening 52 of the sink 44. In addition to being advanced through the pivot nut 48, one end of the lift rod 46 is advanced through the opening 18 of the sleeve portion 16 in a manner facilitating the pivotal attachment of the connecting rod 12 to the lift rod 46. The opposite end of the lift rod 46 is cooperatively engaged to a linkage 54. Such linkage 54 is in turn cooperatively engaged to the lower end of an elongate stem or handle 56. A portion of the handle 56 protrudes upwardly from the sink 44 in close proximity to the associate faucet handles to allow the same to be easily grasped by a user.

In the operation of the drain stopper assembly, due to the pivotal interface of the lift rod 46 to the drain pipe 50 via the pivot nut 48, pulling the handle 56 upwardly in the direction shown by the arrow D in FIG. 1 results in the pivoting of the lift rod 46 in a manner which draws the drain stopper 10 downwardly into the interior of the drain pipe 50. The downward movement of the drain stopper 10 is limited by the engagement of the O-ring 30 of the cap 26 into the complimentary drain opening 52 of the sink 44. Conversely, the application of downward pressure to the handle 56 as results in the movement thereof in a direction opposite to that shown by the arrow D facilitates the pivotal movement of the lift rod 46 in a manner vertically translating the drain stopper 10 and lifting the cap 26 thereof out of its sealed engagement within the drain opening 52. Once the cap 26 is lifted out of sealed engagement within the drain opening 52, water is free to drain from the sink 44 into the drain pipe 50 via the drain opening 52. As such flow occurs, a substantial portion thereof is channeled into the interior of the filter basket 32, and hence over the pellets 42 or other deodorizer disposed within the interior of the filter basket 32. Importantly, the diameter of the upper rim 34 of the filter basket 32 is sized relative to the inner diameter of the drain opening 52 such that a gap of suitable width is defined therebetween so as not to unduly impede the drainage of water from within the sink 44 into the drain pipe 50 via the drain opening 52. The activation of the deodorizer attributable to the periodic passage of water thereover substantially masks or eliminates the emanation of any odors from the drain opening 52.

It is contemplated that from time to time, the pellets 42 or other deodorizer within the interior of the filter basket 32 will need to be replenished. Such replenishment is achieved by simply unscrewing the cap 26 from the connecting rod 12, filling the pellets 42 or other deodorizer into the interior of the filter basket 32, and thereafter reattaching the cap 26 to the connecting rod 12. In typical drain stopper assemblies, the loosening of the pivot nut 48 described above allows the lift rod 46 to be disengaged from the existing drain stopper, thus allowing such existing drain stopper to be removed from within the drain pipe 50 and drain opening 52 of the sink 44. Upon such removal, the drain stopper 10 of the present invention may be advanced into the drain pipe 50 via the drain opening 52, with the lift rod 46 thereafter being re-advanced into the interior of the drain pipe 50 in the above-described manner so as to cooperatively engage the connecting rod 12 of the drain stopper 10 in the manner also discussed above. The retightening of the pivot nut 48 subsequent to the cooperative engagement of the lift rod 46 to the drain stopper 10 completes the retrofit process.

Referring now to FIG. 5, there is shown a drain stopper 10a constructed in accordance with a second embodiment of the present invention. The drain stopper 10a is structurally identical to the above-described drain stopper 10, with the sole distinction between the drain stoppers 10, 10a lying in the addition of a plurality of spike-like projections 58 to the filter basket 32a in the drain stopper 10a. As seen in FIG. 5, the projections 58 protrude outwardly from the filter basket 32a, and are arranged so as to wrap about the filter basket 32 in a spiral-like path. The projections 58 are operable to capture hair passing into the drain pipe 50 via the drain opening 52, thus assisting in the prevention of any undesirable clogging of the P-trap 60 of the drain pipe 50. Thus, in using the drain stopper 10a, it is contemplated that the same will be removed from within the drain opening 52 from time to time to allow for the removal of any hair which accumulates on the projections 58. Those of ordinary skill in the art will recognize that the size, number and/or arrangement of the projections 58 may be varied from that shown in FIG. 5 without departing from the spirit and scope of the present invention.

This disclosure provides exemplary embodiments of the present invention. The scope of the present invention is not limited by these exemplary embodiments. Numerous variations, whether explicitly provided for by the specification or implied by the specification, such as variations in structure, dimension, type of material and manufacturing process may be implemented by one of skill in the art in view of this disclosure.

What is claimed is:

1. A deodorizing drain stopper assembly, comprising:
an elongate connecting rod; and
a filter basket cooperatively engaged to the connecting rod, the filter basket being sized and configured to accommodate a deodorizing element.

2. The drain stopper assembly of claim 1 wherein the filter basket has a generally conical, tapered configuration defining a lower rim which is of a first diameter and an upper rim which is of a second diameter exceeding the first diameter.

3. The drain stopper assembly of claim 2 wherein the connecting rod extends generally axially through the filter basket.

4. The drain stopper assembly of claim 1 further comprising a support frame attached to and extending between the connecting rod and the filter basket.

5. The drain stopper assembly of claim 4 wherein:
the filter basket has a generally conical, tapered configuration defining a lower rim of a first diameter and an upper rim of a second diameter which exceeds the first diameter; and
the upper rim of the filter basket is attached to the support frame.

6. The drain stopper assembly of claim 5 wherein the support frame comprises:
an annular, circularly configured outer wall; and
a plurality of elongate spokes which are integrally connected to the outer wall and extend radially inwardly therefrom;
each of the spokes being attached to the connecting rod, with the upper rim of the filter basket being attached to the outer wall.

7. The drain stopper assembly of claim 6 wherein the spokes of the support frame are integrally connected to the connecting rod such that the connecting rod and the support frame collectively define a unitary structure.

8. The drain stopper assembly of claim 1 wherein the filter basket is fabricated from a mesh-like material.

9. The drain stopper assembly of claim 1 further comprising a cap releasably attached to the connecting rod and operative to enclose the interior chamber of the filter basket when attached to the connecting rod.

10. The drain stopper assembly of claim 1 wherein the deodorizing element comprises a plurality of deodorizing pellets disposed within the interior chamber of the filter basket.

11. The drain stopper assembly of claim 1 wherein the filter basket further comprises a plurality of spike-like projections protruding outwardly therefrom.

12. The drain stopper assembly of claim 11 wherein the projections are arranged in a spiral-like path.

13. A deodorizing drain stopper assembly, comprising:
an elongate connecting rod;
a filter basket cooperatively engaged to the connecting rod and defining an interior chamber; and
a cap releasably attached to the connecting rod and operative to enclose the interior chamber of the filter basket when attached to the connecting rod;
the interior chamber of the filter basket being sized and configured to accommodate a deodorizing element.

14. The drain stopper assembly of claim 13 wherein the filter basket has a generally conical, tapered configuration defining a lower rim which is of a first diameter and an upper rim which is of a second diameter exceeding the first diameter.

15. The drain stopper assembly of claim 14 wherein the connecting rod extends generally axially through the filter basket.

16. The drain stopper assembly of claim 13 further comprising a support frame attached to and extending between the connecting rod and the filter basket.

17. The drain stopper assembly of claim 16 wherein:
the filter basket has a generally conical, tapered configuration defining a lower rim of a first diameter and an upper rim of a second diameter which exceeds the first diameter; and
the upper rim of the filter basket is attached to the support frame.

18. The drain stopper assembly of claim 17 wherein the support frame comprises:
an annular, circularly configured outer wall; and
a plurality of elongate spokes which are integrally connected to the outer wall and extend radially inwardly therefrom;
each of the spokes being attached to the connecting rod, with the upper rim of the filter basket being attached to the outer wall.

19. The drain stopper assembly of claim 18 wherein the spokes of the support frame are integrally connected to the connecting rod such that the connecting rod and the support frame collectively define a unitary structure.

20. The drain stopper assembly of claim 18 wherein the upper rim of the filter basket and a portion of the outer wall of the support frame are received into a complimentary recess defined by the cap when the cap is attached to the connecting rod.
